(19) United States
(12) Patent Application Publication
(10) Pub. No.: US 2023/0285077 A1
(43) Pub. Date: Sep. 14, 2023

(54) PULSED LASER IRRADIATION DEVICE

(71) Applicant: SPECLIPSE INC, Gyeonggi-do (KR)

(72) Inventors: Sung Hyun PYUN, Seoul (KR); Wan Ki MIN, Seoul (KR)

(21) Appl. No.: 17/617,793

(22) PCT Filed: Nov. 11, 2021
(86) PCT No.: PCT/KR2021/016431

§ 371 Date: Dec. 9, 2021

(51) Int. Cl. A61B 18/20; A61B 18/04

(52) U.S. Cl. CPC A61B 18/203; A61B 18/042; A61B 2018/00452

(57) ABSTRACT

A laser irradiation device includes a housing which has an accommodation space formed therein and an opening formed in one side thereof, a laser module which is positioned in the accommodation space and outputs a pulsed beam having a predetermined focal length and a predetermined focal depth range through the opening, a guide member positioned at one side of the housing, and a contact member which is mounted on the guide member and includes a contact portion having a through-hole, through which the pulsed beam passes, formed therein, wherein the contact portion is in contact with a subject to assist in fixing an irradiation point of the pulsed beam, one end of the contact portion is positioned within the focal depth range such that plasma ablation is induced in at least a portion of the subject, and a diameter of the through-hole having a circular shape.

### CROSS-REFERENCE TO RELATED APPLICATIONS

This application is a national phase application of PCT Application No. PCT/KR2021/016431, filed on Nov. 11, 2021. The entire disclosure of the application identified in this paragraph is incorporated herein by reference.

### TECHNICAL FIELD

The present application relates to a pulsed laser irradiation device, and more particularly, to a laser irradiation device including a guide part for adjusting an irradiation distance of a pulsed beam.

### BACKGROUND ART

In the field of modern medical technology, there is an increasing demand for techniques for irradiating a laser onto an object to be analyzed and analyzing spectrum data about light induced therefrom to determine medical information about the object. In addition, in order to determine such medical information, development of a machine learning model for analyzing spectrum data is being actively performed.

In order to improve the accuracy of a machine learning model according to a current technology development trend, a plurality of pieces of light induced to acquire spectrum data for training the machine learning model or a plurality of pieces of light induced to acquire spectrum data to be input to the machine learning model need to have uniform properties.

However, there is a problem in that the conventional laser irradiation device cannot maintain a constant irradiation distance of a pulsed beam irradiated onto an object. Furthermore, since an irradiation distance of a pulsed beam varies whenever the pulsed beam output from the same device is irradiated, a plurality of pieces of light obtained therefrom do not accurately reflect the properties of an object. Therefore, in order to improve the accuracy of a machine learning model, there is a need for a laser irradiation device capable of inducing light having uniform properties from a target whenever a pulsed beam is irradiated.

### SUMMARY

### Technical Problem

The present application is directed to providing a laser irradiation device for acquiring spectrum data including uniform properties from each target even when a pulsed beam is irradiated onto different targets a plurality of times from the laser irradiation device.

The present application is directed to providing a laser irradiation device including a part for assisting in determining an irradiation distance of a pulsed beam such that a user can specify the irradiation distance of the pulsed beam to a subject to which the pulsed beam is to be irradiated.

### Technical Solution

According to an aspect of the present application, there is provided a laser irradiation device for inducing plasma ablation in skin that includes a housing which has an accommodation space formed therein and an opening formed in one side thereof, a laser module which is positioned in the accommodation space and outputs a pulsed beam having a predetermined focal length and a predetermined focal depth range through the opening, a guide member positioned at one side of the housing, and a contact member which is mounted on the guide member and includes a contact portion having a through-hole, through which the pulsed beam passes, formed therein, wherein the contact portion is in contact with a subject to assist in fixing an irradiation point of the pulsed beam, one end of the contact portion is positioned within the focal depth range such that plasma ablation is induced in at least a portion of the subject, and a diameter of the through-hole having a circular shape is formed to be less than or equal to a preset length such that the at least a portion of the subject is maintained within the focal depth range even when the contact member presses the subject.

According to another aspect of the present application, there is provided a contact member, which is used in a laser irradiation device for inducing plasma ablation in skin, that includes a contact portion which has an opening having a circular shape, through which a pulsed beam, which is output from the laser irradiation device and has a preset focal length and a preset focal depth range, passes, and a connection portion connected to the contact portion and connected to one end of the laser irradiation device, wherein the contact portion is mounted at the one end of the laser irradiation device and is in contact with a subject to assist in fixing an irradiation point of the pulsed beam, one end of the contact portion is positioned within the focal depth range such that plasma ablation is induced in at least a portion of the subject when the contact portion is mounted on the laser irradiation device, and a diameter of the opening is formed to be less than or equal to a preset length such that the at least a portion of the subject is maintained within the focal depth range even when the contact member presses the subject.

According to still another aspect of the present application, there is provided a method of inducing plasma ablation using a laser irradiation device including a laser module configured to output a pulsed laser having a preset focal length and a preset focal depth range through an opening formed in a housing, and a contact member including a contact portion which has a through-hole having a circular shape, through which the pulsed laser passes, formed therein, the contact portion being disposed within the focal depth range, the method including bringing the contact portion into contact with a first subject, irradiating a pulsed beam onto the first target, receiving light induced in the first target, bringing the contact portion into contact with a second subject, irradiating the pulsed beam onto a second target, and receiving light induced in the second target, wherein, in order for a position of the first target lifted by pressure when the contact portion is in contact with the first subject and a position of the second target lifted by pressure when the contact portion is in contact with the second subject to be maintained within the focal depth range, a diameter of the through-hole is formed to be less than or equal to a preset length.

### Advantageous Effects

According to the present application, there can be provided a laser irradiation device in which an irradiation distance of a pulsed beam from the laser irradiation device to a target is set within a predetermined range in order to acquire spectrum data including uniform properties from a target.

According to the present application, there can be provided a laser irradiation device including a guide part that is in contact with a target to assist a user in determining an irradiation distance of a pulsed beam.

### BRIEF DESCRIPTION OF THE DRAWINGS

FIG. 1 illustrates an example of a spectrum analysis system according to one embodiment.

FIG. 2 shows induction conditions of plasma ablation according to one embodiment.

FIG. 3 illustrates a spot size according to an irradiation distance of a pulsed beam irradiated from a laser irradiation device according to one embodiment.

FIG. 4 illustrates problems of a laser irradiation device according to a related art.

FIG. 5 illustrates an exterior of a laser irradiation device according to one embodiment.

FIG. 6 illustrates an internal configuration of the laser irradiation device according to one embodiment.

FIG. 7 is an upper perspective view of a contact member according to one embodiment.

FIG. 8 is a lower perspective view of the contact member according to one embodiment.

FIG. 9 is a cross-sectional view of the contact member according to one embodiment.

FIG. 10 illustrates a coupling structure of a guide part of the laser irradiation device according to one embodiment.

FIG. 11 illustrates the contact member in contact with a subject being observed in a direction in which a pulsed beam is irradiated according to one embodiment.

FIG. 12 illustrates a path of a pulsed beam output from the laser irradiation device according to one embodiment together with a configuration of the laser irradiation device.

FIG. 13 illustrates an irradiation path of a pulsed beam when there is no target in one embodiment.

FIG. 14 illustrates an irradiation path of a pulsed beam when the contact member presses a target in one embodiment.

FIG. 15 shows spectrum data according to a diameter of a through-hole and pressure in one embodiment.

FIG. 16 illustrates a method of generating induced light in a single target using a laser irradiation device according to one embodiment.

FIG. 17 illustrates a method of generating induced light in a plurality of targets using a laser irradiation device according to one embodiment.

### DETAILED DESCRIPTION

The above-described objects, characteristics, and advantages of the present invention will be made more apparent by the following detailed description with reference to the accompanying drawings. Since the present invention may be variously modified and have various embodiments, specific embodiments will be shown in the accompanying drawings and described in detail in a detailed description.

Embodiments described in this specification are made to clearly describe the scope of the present invention to those having ordinary skill in the art and are not intended to limit the present invention. It should be interpreted that the present invention may include substitutions and modifications within the technical scope of the present invention.

In the present specification, the accompanying drawings are to facilitate the description of the present invention, and the shape in the drawings may be exaggerated for the purpose of convenience of description so that the present invention should not be limited to the drawings.

Moreover, detailed descriptions about well-known functions or configurations associated with the present invention will be ruled out in order to not unnecessarily obscure the essence of the present invention. It should also be noted that ordinal numbers (such as first and second) used in the description of the preset specification are used only to distinguish one component from another component.

In addition, the terms “unit,” “module,” and “parts” for components used in the following description are given or used interchangeably only for facilitation of preparing this specification, and thus they are not granted a specific meaning or function.

According to one embodiment of the present application, a laser irradiation device for inducing plasma ablation in skin includes a housing which has an accommodation space formed therein and an opening formed in one side thereof, a laser module which is positioned in the accommodation space and outputs a pulsed beam having a predetermined focal length and a predetermined focal depth range through the opening, a guide member positioned at one side of the housing, and a contact member which is mounted on the guide member and includes a contact portion having a through-hole, through which the pulsed beam passes, formed therein, wherein the contact portion is in contact with a subject to assist in fixing an irradiation point of the pulsed beam, one end of the contact portion is positioned within the focal depth range such that plasma ablation is induced in at least a portion of the subject, and a diameter of the through-hole having a circular shape is formed to be less than or equal to a preset length such that the at least a portion of the subject is maintained within the focal depth range even when the contact member presses the subject.

The diameter of the through-hole may be set such that the subject is maintained to be 3% or less of the focal length even when the contact member presses the subject.

The diameter of the through-hole may be set such that the subject protrudes by 1 mm or less when the contact member presses the subject.

The diameter of the through-hole may be 7 mm or less.

The diameter of the through-hole may be 3 mm or less.

The diameter of the through-hole may be greater than a spot size of the pulsed beam.

The focal depth range may be defined as a preset range formed from a focus of the pulsed beam along an axis of an irradiation direction of the pulsed beam, and one end of the contact portion may be positioned within a half range of the focal depth range based on the focus.

One end of the contact portion may be positioned at the focus.

The contact member may have a thickness of 0.5 mm to 1.5 mm.

A distance from one end of the guide member to a distal end of the contact member may be defined as a first length, a distance between the opening and a start point from which the pulsed beam is output may be defined as a second length, and the sum of the first length and the second length may be set to be greater than or equal to the focal distance of the pulsed beam so that, even when the guide member and the housing are coupled to overlap each other, the contact portion may be maintained within the focal depth range.

The contact member may be made of a light-transmitting material.

The contact member may be formed to be detachable from and attachable to the guide member.

The contact member may include a sidewall on which a connection portion is formed, and the connection portion may be coupled to the guide member in a snap-fit form.

According to another embodiment, a contact member, which is used in a laser irradiation device for inducing plasma ablation in skin, includes a contact portion which has an opening having a circular shape, through which a pulsed beam, which is output from the laser irradiation device and has a preset focal length and a preset focal depth range, passes, and a connection portion connected to the contact portion and connected to one end of the laser irradiation device, wherein the contact portion is mounted at the one end of the laser irradiation device and is in contact with a subject to assist in fixing an irradiation point of the pulsed beam, one end of the contact portion is positioned within the focal depth range such that plasma ablation is induced in at least a portion of the subject when the contact portion is mounted on the laser irradiation device, and a diameter of the opening is formed to be less than or equal to a preset length such that the at least a portion of the subject is maintained within the focal depth range even when the contact member presses the subject.

According to still another embodiment, as a method of inducing plasma ablation using a laser irradiation device including a laser module configured to output a pulsed laser having a preset focal length and a preset focal depth range through an opening formed in a housing, and a contact member including a contact portion which has a through-hole having a circular shape, through which the pulsed laser passes, formed therein, the contact portion being disposed within the focal depth range, there may be provided a method of inducing plasma ablation using a laser irradiation device, the method including bringing the contact portion into contact with a first subject, irradiating a pulsed beam onto the first target, receiving light induced in the first target, bringing the contact portion into contact with a second subject, irradiating the pulsed beam onto a second target, and receiving light induced in the second target, wherein, in order for a position of the first target lifted by pressure when the contact portion is in contact with the first subject and a position of the second target lifted by pressure when the contact portion is in contact with the second subject to be maintained within the focal depth range, a diameter of the through-hole is formed to be less than or equal to a preset length.

The present application relates to a laser irradiation device. The laser irradiation device of the present application is directed to irradiating a pulsed beam onto an object to induce light in the object irradiated with the pulsed beam. Information about light induced from the object may be analyzed through a data analysis device connected to the laser irradiation device in various manners. The data analysis device may acquire spectrum data about light induced from the object and may identify the properties of the object from the spectrum data of the object through various spectroscopic analysis techniques.

The laser irradiation device according to one embodiment may induce light usable in various spectroscopic analysis techniques from an object. As an example, the laser irradiation device may induce scattered light usable in Raman spectroscopy from an object. As another example, the laser irradiation device may induce fluorescence light usable in fluorescence analysis from an object. As still another example, the laser irradiation device may induce plasma ablation in an object in order to induce light usable in laser-induced breakdown spectroscopy (hereinafter referred to as “LIBS”). In addition, the laser irradiation device of the present application may be used to induce light usable in various known spectroscopic analysis techniques. However, in the following description of the present specification, for convenience of description, a case in which the laser irradiation device according to one embodiment is used to induce light used in the LIBS will be mainly described.

Hereinafter, an object irradiated with a pulsed beam, that is, a subject, which is an object to which a pulsed beam is applied to generate induced light, will be referred to as a “target.” In addition, a target may refer to an object to be subjected to spectrum analysis. A main body including a “target” will be referred to as a “subject.” That is, the “target” is a partial area of the “subject” and may be understood as a portion of the subject onto which a pulsed beam is to be irradiated by the laser irradiation device. In addition, an area of the subject and/or target onto which a pulsed beam is irradiated may be referred to as an “irradiation area.”

In the present specification, there may be various subjects. For example, when a disease of a patient is diagnosed or the presence or absence of an abnormality of a subject to be analyzed is determined, the subject may include portions of components constituting of a body of the patient, such as skin, internal and external tissues of the body, various cells, blood, and saliva. In addition, the target may refer to a partial area of the subject such as tissue suspected of being a lesion present in skin, and when the subject is blood or cells, the target may be substantially the same as the subject. Therefore, it will be understood that the terms “target” and “subject” may be used interchangeably in the following description of the present specification.

Hereinafter, the contents to be disclosed by the present application will be described in detail with reference to the accompanying drawings.

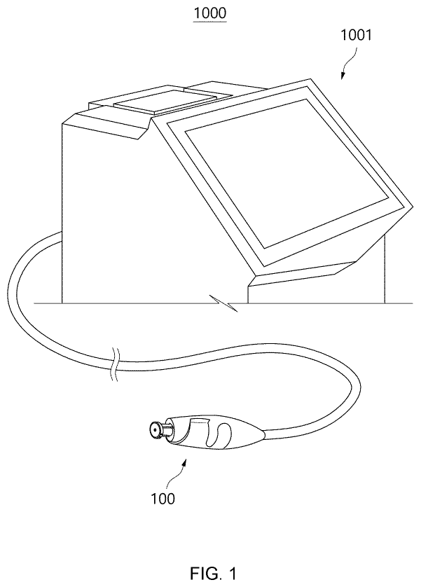

FIG. 1 illustrates an example of a spectrum analysis system according to one embodiment.

Referring to FIG. 1, a spectrum analysis system 1000 may be provided. The spectrum analysis system 1000 according to one embodiment may include a laser irradiation device 100 and a data analysis device 1001.

In the spectrum analysis system 1000, the laser irradiation device 100 may irradiate a pulsed beam to at least a portion of a target to induce light therefrom, and the data analysis device 1001 may analyze a spectrum of the induced light.

Here, the laser irradiation device 100 may irradiate the pulsed beam onto the target. Plasma ablation may be induced in at least a partial area of the target onto which the pulsed beam is irradiated. In this case, light induced due to the plasma ablation may be generated in the target in which the plasma ablation is induced. In the following description of the present specification, light induced due to plasma ablation will be referred to as “induced light.” That is, the laser irradiation device 100 may generate induced light due to plasma ablation by irradiating a laser onto the target. For example, the induced light due to the plasma ablation may include light according to plasma emission and light according to element specific emission.

The data analysis device 1001 may receive the induced light. Here, the induced light may be collected by the laser irradiation device 100 and transmitted to the data analysis device 1001 through a separate optical structure.

The data analysis device 1001 may spectroscopically analyze the induced light to acquire spectrum data. To this end, the data analysis device 1001 may include a separate spectrometer. Alternatively, the spectrometer may be integrated into the laser irradiation device 100, and the data analysis device 1001 may receive spectrum data about the induced light from the laser irradiation device 100.

The data analysis device 1001 may analyze the spectrum data to determine medical information related to the target. For example, the medical information may refer to the presence or absence of a diseased tissue.

The data analysis device 1001 may include a separate processor capable of operating data and a memory in which algorithms or programs for data analysis are stored. Accordingly, the processor of the data analysis device 1001 may analyze the spectrum data using various algorithms or programs stored in the memory and may determine the medical information therefrom.

Here, the data analysis device 1001 may use technologies such as big data and artificial intelligence to analyze the spectrum data. For example, the data analysis device 1001 may analyze the spectrum data using a pre-trained machine learning model to acquire the medical information.

The machine learning model according to one embodiment may be trained to determine whether a diseased tissue is present in a subject to be analyzed. To this end, the machine learning model may be trained using spectrum data acquired from various subjects. Specifically, the machine learning model may be trained based on learning data in which each piece of medical information is labeled in spectrum data acquired from a subject about which medical information (for example, the presence or absence of a diseased tissue) is known in advance.

When the machine learning model is trained based on a plurality of pieces of spectrum data, the output accuracy of the machine learning model may be improved so that the plurality of pieces of spectrum data are acquired under similar conditions. Spectrum data is information about light, and the accuracy of the machine learning model may differ greatly even when there is a minute difference in information included in light. For example, when the machine learning model is trained with spectrum data about plasma light induced due to plasma ablation, the uniformity of the plasma ablation that is the basis of learning data may have a great influence on the accuracy of the machine learning model. Therefore, in order to improve the accuracy of the machine learning model, it may be important to control the uniformity of plasma ablation in a target 1 due to a pulsed beam 5 irradiated by the laser irradiation device 100.

Hereinafter, conditions of plasma ablation induced from the laser irradiation device 100 according to one embodiment will be described.

In one embodiment of the present application, a pulsed beam irradiated from the laser irradiation device 100 should be able to induce plasma ablation in at least a portion of a target. Here, the plasma ablation is related to power per unit area (hereinafter referred to as “power density”) and/or energy per unit area (hereinafter, referred to as “fluence”) which is applied to an irradiation area of a target by the pulsed beam.

Exemplarily, when a pulsed beam is applied to a target, power density and fluence may be as follows.

power density=energy per pulse/(pulse width×irradiation area) [Equation 1]

The power density may refer to energy per unit area which is applied to the target per unit time. That is, as shown in Equation 1, power density of a pulsed laser may be a value obtained by dividing power, which is obtained by dividing laser pulse energy by a pulse width, by an irradiation area. Here, the irradiation area may refer an area of the pulsed beam incident on the target, that is, an area of an irradiation area.

fluence=energy per pulse/irradiation area [Equation 2]

The fluence may refer to energy per unit area which is applied to the target. That is, as shown in Equation 2, fluence of a pulsed laser may be a value obtained by dividing laser pulse energy by an irradiation area.

fluence=power density×pulse width [Equation 3]

Accordingly, as shown in Equation 3, fluence of a pulsed laser may be a value obtained by multiplying power density of the pulsed laser by a pulse width, and the power density of the pulsed laser may be a value obtained by dividing the fluence of the pulsed laser by the pulse width.

Here, when a laser is irradiated onto a target using a pulsed beam, the formation of plasma is related to a power density of a pulsed laser. Specifically, when a sufficient power density is applied to the target, plasma ablation may be generated in the target.

Hereinafter, for convenience of description, the minimum power density that should be applied to induce plasma ablation in a target will be referred to as an ablation threshold Aₜₕ.

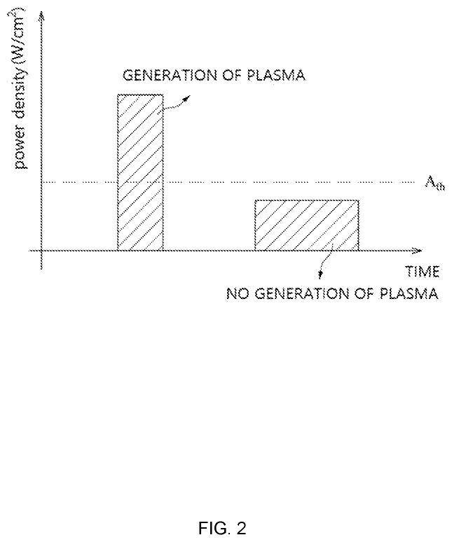

Referring to FIG. 2, various power densities and fluences may be applied to a target according to one embodiment of the present invention.

Referring to FIG. 2, a power density and fluence according to a time for which a pulsed beam is irradiated, that is, according to a pulse width, are shown.

For example, referring to FIG. 2, when a power density greater than or equal to an ablation threshold Aₜₕ is applied to a target, plasma ablation may be induced in the target.

For another example, referring to FIG. 2, when a power density less than or equal to the ablation threshold Aₜₕ is applied to the target, plasma ablation may not be induced in the target.

That is, when a sufficient power density is applied to the target, plasma ablation may be generated in the target. For example, when the spectrum analysis system 1000 determines the presence or absence of skin cancer in skin suspected of having skin cancer, plasma ablation may be induced in epidermis of the skin only when a sufficient power density is applied to the skin. Thus, spectrum data about light induced from the plasma ablation may be analyzed to determine the presence or absence of the skin cancer.

Here, the ablation threshold Aₜₕ may have a different value according to a type or state of the target. For example, when the target is a part of a body of a human or animal, plasma ablation may be induced in the target when a power density applied to the target according to laser irradiation is 0.1 GW/cm² or more.

Meanwhile, according to one embodiment of the present specification, the spectrum analysis system 1000 may adjust power density and fluence values so as to not damage a target while inducing plasma ablation in the target for safe and accurate diagnosis. For example, the laser irradiation device 100 may adjust the energy, pulse width, and irradiation area of a laser irradiated onto the target to allow an intensity of a laser per unit area irradiated onto the target to be 0.1 GW/cm² or more and a magnitude of energy per unit area to be 40 J/cm² or less. For example, when the above-described fluence and power density are applied to skin, plasma ablation may be generated only in epidermis so that a non-destructive test can be substantially performed without damage to body tissues such as blood vessels.

Hereinafter, according to one embodiment of the present specification, a setting method and a settable value range of the above-described energy, pulse width, irradiation area, and the like of the laser according to specifications of a device, an apparatus, or equipment will be described.

The laser irradiation device 100 according to one embodiment may adjust a type of a laser-activating medium and energy applied to the laser-activating medium to set energy and a pulse width of a generated pulsed beam. For example, the laser irradiation device 100 may generate a pulsed beam having an energy of about 10 mJ to 100 mJ per pulse and a pulse width of about 1 ps to 1 ms.

In addition, an irradiation area of a pulsed beam may be changed due to a distance between the laser irradiation device 100 and a target. That is, the laser irradiation device 100 may set an irradiation distance of a pulsed beam to change or adjust an irradiation area of the pulsed beam applied to the target. For example, as the distance between the laser irradiation device 100 and the target is increased, the irradiation area may be widened, and as the target approaches a laser focus according to the irradiation distance, the irradiation area may become smaller.

A pulsed beam output by the laser irradiation device 100 may have a spot size. Here, the spot size may refer to a diameter of the pulsed beam according to an irradiation distance of the pulsed beam. That is, when a pulsed beam is applied to the target, it may be expressed that a spot size of the pulsed beam applied to the target is determined according to a distance between the laser irradiation device 100 and the target, and a diameter of an irradiation area of the pulsed beam applied to the target corresponds to the spot size. Accordingly, the spot size and the diameter of the irradiation area have different perspectives but have substantially the same meaning, and the above terms may be used interchangeably herein.

When an irradiation area is set by a distance between the laser irradiation device 100 and the target, a power density and fluence may be considered. Specifically, an irradiation area of a pulsed beam irradiated to the target may have a diameter of 1 um to 10 mm or may be set to have an area in a range of 0.7 um² to 70 mm². For example, preferably, the irradiation area has a diameter of 100 μm to 5 mm or an area of 0.01 mm² to 20 mm².

Meanwhile, since the above-described ranges of the intensity, energy per pulse, pulse width, and irradiation area of the laser are merely an example, the embodiments of the present specification are not limited thereto.

As described above, the data analysis device 1001 may analyze spectrum data of a target using the machine learning model trained based on spectrum data acquired in advance from various subjects. When the machine learning model is trained, the accuracy of the machine learning model may be improved when a set of pieces of training data set used for learning is a set of pieces of learning data acquired under relatively uniform conditions. In addition, even when the trained machine learning model is used, the output accuracy of the machine learning model may be improved when input data acquired under conditions similar to those of learning data of the machine learning model is input.

In such a situation, the spectrum analysis system 1000 according to one embodiment needs to be trained using spectrum data acquired under uniform conditions, and furthermore, it may be essential that spectrum data acquired under conditions as similar as possible to those of learning data is used as input data for the machine learning model.

However, when several parameters (for example, energy per pulse, and a pulse width) of the laser irradiation device 100 are set as described above, a diameter or area of an irradiation area of a pulsed beam is determined according to an irradiation distance of a laser. In addition, a power density or fluence applied to a target may be changed according to an irradiation area or diameter of a pulsed beam. When the power density or fluence applied to the target is changed, the properties of induced light induced due to plasma ablation induced in the target may be changed, and sometimes, the plasma ablation may not be induced.

In other words, it may be expressed that, as an irradiation distance of a pulsed beam irradiated from the laser irradiation device 100 becomes more uniform, the properties of induced light induced by a pulsed beam may be uniform. In addition, as the properties of the induced light become more uniform, it means that the learning accuracy of the machine learning model or the accuracy of output data can also be improved.

To this end, in the following description of the present specification, a preferred irradiation distance of a pulsed beam irradiated from the laser irradiation device according to one embodiment will be described with reference to the drawings.

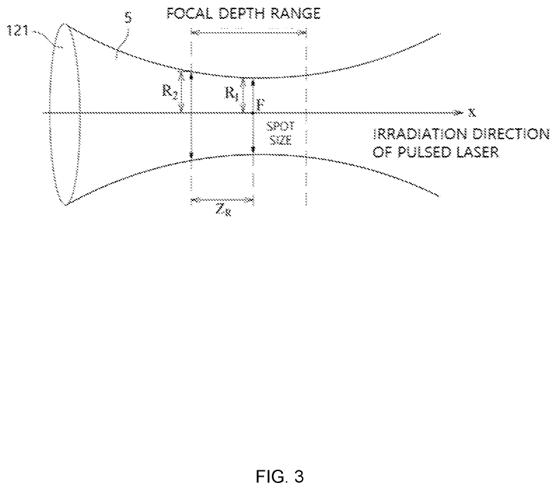

FIG. 3 illustrates a spot size according to an irradiation distance of a pulsed beam irradiated from the laser irradiation device according to one embodiment.

In the laser irradiation device 100 according to one embodiment, various spot sizes may be set according to irradiation distances. That is, the spot size may be set according to an irradiation distance at which a pulsed beam is irradiated onto a target. Here, in addition to the spot size, an intensity or pulse width of the irradiated pulsed beam is regarded as being preset in the laser irradiation device 100 or other external apparatuses, unless otherwise specified.

Referring to FIG. 3, a pulsed beam 5 may have a focus F. As will be described below, the laser irradiation device 100 according to one embodiment may include a light adjustment member 121. Here, the light adjustment member 121 may be implemented as a lens. In addition, the pulsed beam may be irradiated in a form converging to a center of a traveling direction thereof due to a refractive index or spherical aberration of the light adjustment member 121. Here, the focus F may refer to a position, which is on a traveling path of the pulsed beam 5, at which a diameter of the pulsed beam 5, that is, a spot size, is the smallest. After passing through the focus, the pulsed beam 5 may travel again in a form that goes away from the center of the traveling direction. In this case, an irradiation shape of the pulsed beam 5 may have a substantially symmetrical shape with respect to the focus. In addition, a distance to the focus F from a start point from which the pulsed beam 5 is irradiated may be referred to as a focal length.

In addition, when a radius of the pulsed beam 5 at the focal length, that is, a radius of the spot size, is R₁, a distance ZR from the focus F of the pulsed beam 5 to a point, at which the radius of the pulsed beam 5 becomes R2 with respect to an axis x of an irradiation direction of a pulsed laser, may be expressed as a Rayleigh length. Here, R2 may have a predetermined ratio with R₁. In addition, there may be various methods of determining R2. For example, R2 may be determined to be √2 times R₁. However, in the present invention, an example of a case in which R2 is determined to be √2 times R₁ will be described, but this is only for convenience of description, and the spirit of the present specification is not limited thereto.

In addition, an area formed up to the Rayleigh length ZR in both directions of the focus along the axis x along which the pulsed beam 5 is irradiated may be expressed as a focal depth range. As described above, a power density applied to a target may vary according to the spot size of the pulsed beam 5, which has a great influence on plasma ablation being induced or induced light being generated due to the plasma ablation. In principle, a focal depth range may be determined based on a focus, but in the following description of the present specification, for convenience of description, a focal depth range may also be expressed as a specific range calculated from a start point from which a pulsed beam is output.

Therefore, in order for plasma ablation to be uniformly induced by the laser irradiation device 100, it is necessary to minimize a change in spot size of the pulsed beam 5 applied to the target, and to this end, it is necessary to maintain an irradiation distance of the pulsed beam 5 to the target constant. In other words, it is necessary to maintain an end point of an irradiation path of the pulsed beam 5 constant, and specifically, it is preferable that the end point of the irradiation path of the pulsed beam 5 is maintained within the focal depth range.

Prior to describing specific embodiments of the present application, problems of a laser irradiation device according to a related art will be described with reference to the drawings.

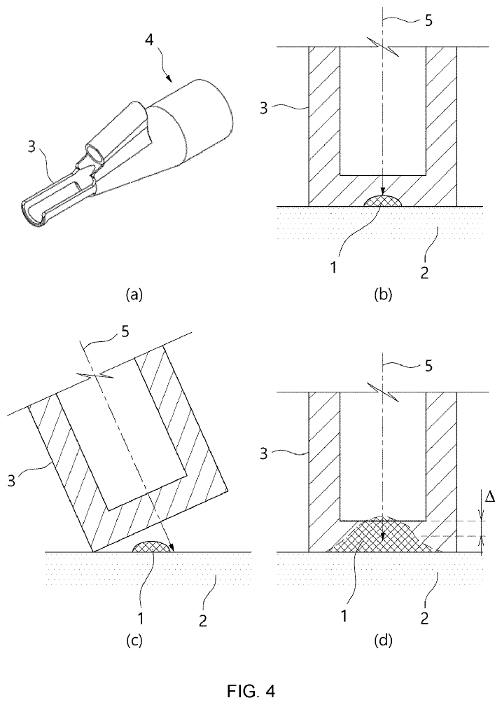

FIG. 4 illustrates the problems of the laser irradiation device according to the related art.

Referring to FIG. 4A, the laser irradiation device according to the related art generally includes a separate tip 4 mounted on the laser irradiation device. In addition, the tip 4 includes a guide frame 3 for guiding an irradiation position of a pulsed beam 5. In general, in the laser irradiation device according to the related art, a lower end portion of the guide frame 3 has a ring shape or a semicircular shape. During use, the lower end portion of the guide frame 3 is disposed to be positioned in a peripheral area of a target 1, and the pulsed beam 5 is irradiated onto a partial area in the lower end portion of the guide frame 3 and irradiated onto the target 1. In this case, the guide frame 3 may be placed on one area of a subject 2 including the peripheral area of the target 1.

When the pulsed beam 5 is irradiated in a normal use mode as shown in FIG. 4B, plasma ablation may be induced in the target 1.

However, since, during use, the laser irradiation device according to the related art cannot clearly guide a degree by which a user presses the subject 2 through an angle between the subject 2 and the laser irradiation device or the laser irradiation device, a case may occur in which plasma ablation cannot be induced in the target 1 during use.

For example, FIG. 4C is a view for describing a case in which, when the user uses the laser irradiation device according to the related art, the angle between the laser irradiation device and the subject 2 is an unintended angle. Referring to FIG. 4C, when the user uses the laser irradiation device according to the related art, a case may occur in which a portion of the guide frame 3 is spaced apart from the subject 2, and thus the pulsed beam 5 is not irradiated onto the target 1. Alternatively, even when the pulsed beam 5 is irradiated onto the target, an irradiation distance of the pulsed beam 5 may be changed, and thus the target 1 may deviate from a focal depth range. Accordingly, even when the pulsed beam 5 is irradiated onto the target 1, plasma ablation may not be induced in the target. In particular, when the laser irradiation device according to the related art includes the guide frame 3 having the semicircular shape, the laser irradiation device is inclined to an open area during use, and thus the possibility the target 1 deviating from the focal length range is increased.

FIG. 4D is a view for describing a case in which, when the user uses the laser irradiation device according to the related art, the laser irradiation device presses the subject 2 to an unintended degree. Referring to FIG. 4D, when the user uses the laser irradiation device according to the related art, the guide frame 3 of the laser irradiation device is brought into contact with the subject 2. When the user presses the guide frame 3 of the laser irradiation device toward the subject 2 more than is necessary, the target 1 and a portion of the subject 2 protrude in an irradiation direction of a laser due to elasticity of the subject 2. Accordingly, even when the pulsed beam 5 is irradiated onto the target, an irradiation distance of the pulsed beam 5 may be decreased, and thus the target 1 may deviate from the focal depth range. In this case, even when the pulsed beam 5 is irradiated onto the target 1, plasma ablation may not be induced in the target 1. In addition, although not shown, even when the user uses the guide frame 3 of the laser irradiation device according to the related art in a form of not being brought into contact with the subject 2, plasma ablation may not be induced in the target 1.

In order to solve the above problems of the related art and induce uniform plasma ablation, there may be provided a laser irradiation device 100 in which an irradiation point of a pulsed beam 5 can be specified according to one embodiment of the present application. In addition, there may be provided the laser irradiation device 100 in which the irradiation distance of the pulsed beam 5 can be specified within a preset range.

Hereinafter, the laser irradiation device 100 according to the embodiment of the present application will be described with reference to drawings.

First, a structural configuration of the laser irradiation device 100 according to one embodiment of the present application will be described with reference to the drawings.

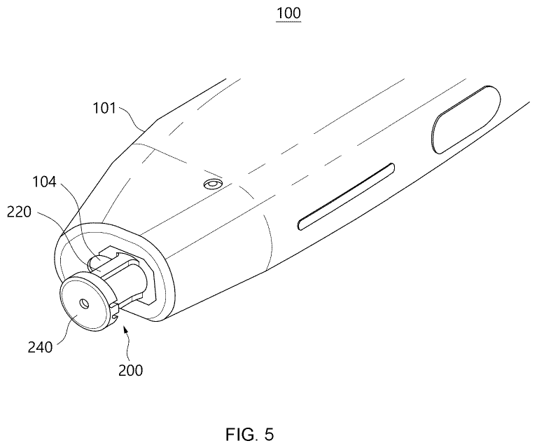

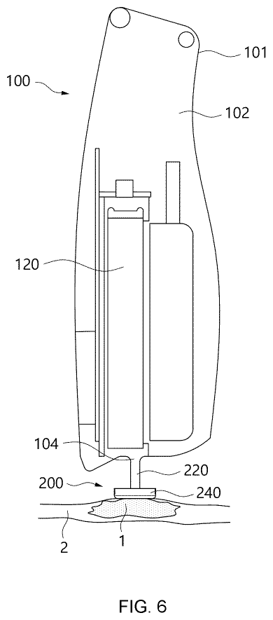

FIGS. 5 and 6 illustrate an overall configuration of the laser irradiation device 100 according to one embodiment. Specifically, FIG. 5 illustrates an exterior of the laser irradiation device 100 according to one embodiment, and FIG. 6 illustrates an internal configuration of the laser irradiation device 100 according to one embodiment.

The laser irradiation device 100 according to one embodiment may irradiate a pulsed beam onto a target 1. Specifically, the laser irradiation device 100 may irradiate the pulsed beam onto the target 1 to induce plasma ablation in at least a portion of the target 1 and may induce induced light due to the plasma ablation.

Hereinafter, descriptions will be provided with reference to FIGS. 5 and 6 together. The laser irradiation device 100 includes a housing 101 and a guide part 200. The housing 101 may form an exterior of the laser irradiation device 100.

The housing 101 may be gripped by a user when the laser irradiation device 100 is used. In addition, a separate switch may be included in the housing 101. The user may grip the housing 101 and operate the switch formed on the housing 101 to irradiate a pulsed beam.

The housing 101 may include an opening 104. The laser irradiation device 100 may irradiate a pulsed beam 5 through the opening 104. In addition, the housing 101 may further include a separate aperture for receiving induced light due to plasma ablation. The induced light received from the aperture may be transmitted to a data analysis device 1001 through a separate optical structure so as to acquire spectrum data.

The guide part 200 may be disposed at one side of the housing 101. Specifically, the guide part 200 may be disposed near the opening 104 to assist the user in determining a position at which the pulsed beam is irradiated. At least a portion of the guide part 200 may be formed to extend from one side of the housing. In addition, the guide part 200 may be formed as a separate member and mounted at one side of the housing 101.

The guide part 200 may include a support member 220 and a contact member 240. The support member 220 may be disposed at one side of the housing 101 to perform a function of determining an irradiation distance of the pulsed beam irradiated from the laser irradiation device 100. The support member 220 may be integrated with the housing 101 and formed to extend from one side of the housing 101 or may be mounted at one side of the housing 101 as a separate member. Here, the support member 220 may extend in a direction in which the pulsed beam is irradiated. For example, the support member 220 may extend along a virtual axis set to be parallel to the direction in which the pulsed beam is irradiated.

The support member 220 may include one or more support structures. Preferably, the support member 220 may include two support structures. The support structures may be formed to be spaced apart from each other to define an open area.

Since the support member 220 includes the open area, the user can visually see whether the contact member 240 is positioned at an intended position.

The contact member 240 may be positioned at one side of the support member 220. The contact member 240 may be in contact with the target and/or subject to determine an irradiation position of the pulsed beam. In addition, the contact member 240 may determine an irradiation distance of the pulsed beam irradiated from the laser irradiation device 100 together with the support member 220. That is, the contact member 240 may have a predetermined thickness, and the thickness of the contact member 240 and a length of the support member 220 may be combined to determine the irradiation distance of the pulsed beam. This will be described below in detail.

The contact member 240 may be formed integrally with the support member 220. Alternatively, the contact member 240 may be formed as a separate member to be implemented in a form that is detachably mounted at one side of the support member 220. When a subject 2 is skin of a human or animal and the target 1 is one area of the subject 2 suspected of being a lesion tissue, it is preferable that the contact member 240 is formed as a disposable contact member for health purposes.

In addition, a laser generation module 120 may be mounted in an accommodation portion 102 inside the housing 101. The laser generation module 120 may output a pulsed beam or a continuous beam according to a laser-activating medium. However, in the description of the present specification, for convenience of description, the pulsed beam will be mainly described.

Here, when a pulsed laser is output, a laser generated by the laser-activating medium may be excited with a pulsed signal, or Q switching, mode synchronization, or the like may be used. A pulse width (duration) may be adjusted to adjust an output intensity (energy per unit time) through the laser.

An example of parameters of the laser irradiation device 100 according to one embodiment is as follows.

Table 1 shows allowable ranges of various parameters of the laser irradiation device 100 according to one embodiment.

TABLE 1

| Parameter | Range |
|---|---|
| Diameter of spot size (mm) | 0.001 to 10 |
| Power density (GW/cm²) | >0.1 |
| Fluence (J/cm²) | <40 |
| Energy per pulse (mj) | 10 to 100 |
| Pulse width (ns) | 0.001 to 1,000 |
| Wavelength range (nm) | 200 to 1,000 |
| Emission period (ms) | <1,000 |

The parameters disclosed in Table 1 are values set to induce plasma ablation in the target as described above with reference to FIG. 2.

Referring to FIG. 6 again, the laser generation module 120 may change a shape of the pulsed beam. Here, the shape of the pulsed beam may include a collimated beam, a focused beam, and a defocused beam.

When a shape of a laser is changed, an irradiation area of a laser irradiated onto the target may be determined. Accordingly, an intensity of energy applied to the target by the laser may be determined.

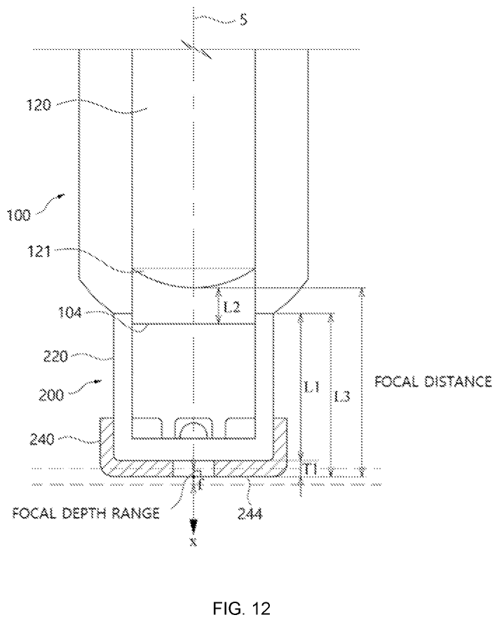

Here, the laser generation module 120 may be provided together with an optical member 121 (see FIG. 12) implemented using optical elements such as a lens, a filter, a mirror, and a pinhole in order to change the properties of the pulsed beam. This will be described below.

In the following description, the detailed structure and function of the laser irradiation device 100 according to the embodiment, in particular, the guide part 200, will be described with reference to drawings.

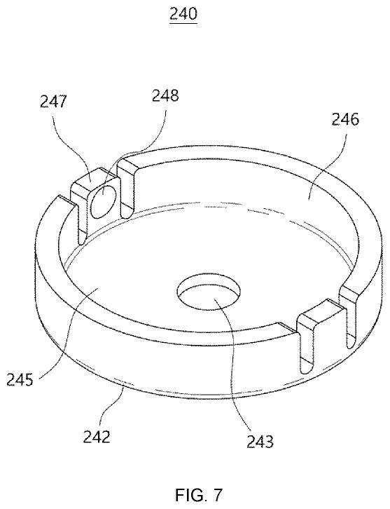

First, the contact member 240 will be described with reference to FIGS. 7 to 9.

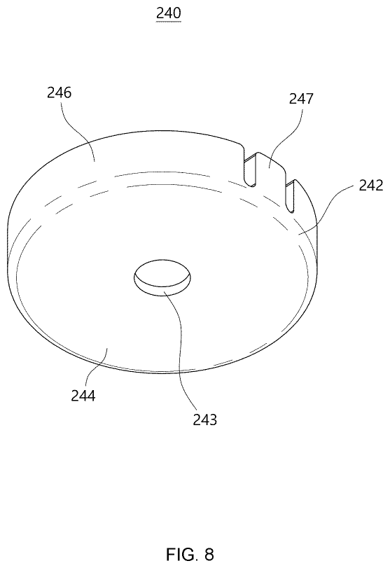

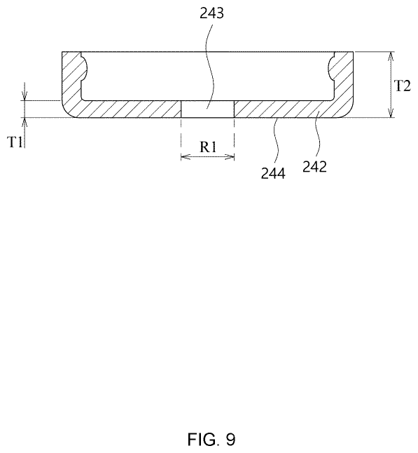

FIG. 7 is an upper perspective view of the contact member 240 according to one embodiment, FIG. 8 is a lower perspective view of the contact member 240 according to one embodiment, and FIG. 9 is a cross-sectional view of the contact member 240 according to one embodiment.

Referring to FIGS. 7 to 9, the contact member 240 includes a contact portion 242. The contact portion 242 may be formed in a plate shape. Since the contact portion 242 is formed in the plate shape, the contact portion 242 may be in surface contact with the subject 2 or the target 1. This is merely an example, and the contact portion 242 is not necessarily formed in the plate shape. For example, the contact portion 242 may have a bar shape formed around a through-hole 243 to be described below. That is, the contact portion 242 may be variously designed and changed as long as the contact portion 242 has a shape capable of assisting an irradiation direction or irradiation distance of the pulsed beam 5 in contact with the subject 2 or the target 1 over a predetermined area or more.

In addition, the through-hole 243 may be formed in at least a portion of the contact portion 242. The pulsed beam output from the laser irradiation device 100 may be applied to at least a portion of the target 1 through the through-hole 243. When the contact portion 242 has a circular shape, the through-hole 243 may be formed in a central portion of the contact portion 242 having the circular shape.

When a user uses the laser irradiation device 100 according to the embodiment, after the laser irradiation device 100 is aligned such that the through-hole 243 is positioned on the target 1, the pulsed beam may be output. That is, when the laser irradiation device 100 is used, the through-hole 243 may be disposed at a position corresponding to the target 1 by the user. Thus, an irradiation area of the pulsed beam 5 may be disposed in at least a portion of the target 1. Here, in order for the irradiation area of the pulsed beam 5 to be formed at a central portion of the through-hole 243, the laser irradiation device 100 may be designed such that the pulsed beam 5 passes through the central portion of the through-hole 243. However, this is merely an example, and as long as the pulsed beam 5 can pass through the through-hole 243, the laser irradiation device 100 may be designed such that the pulsed beam 5 passes through any area included in the through-hole 243.

It is preferable that the through-hole 243 has a circular shape, but the present invention is not limited thereto, and the through-hole 243 may have one of various shapes. For example, the through-hole 243 may have a polygonal shape. In addition, a width of the through-hole 243 (for example, a diameter when the through-hole 243 has the circular shape) may be preset. Since the width of the through-hole 243 is preset, even when the contact member 240 presses the target 1 or the subject 2, the target 1 may be positioned within a preset range in an irradiation path of the pulsed beam. This will be described below in detail.

In addition, the contact member 240 may include a sidewall 246 formed around the contact portion 242. As will be described below, the sidewall 246 may be connected to a first connection portion 224 of the support member 220. The sidewall 246 may be formed integrally with the contact portion 242. A connection area between the sidewall 246 and the contact portion 242 may be formed as a curved surface. Since the connection area between the sidewall 246 and the contact portion 242 is formed as the curved surface, when the sidewall 246 and the contact portion 242 are in contact with the subject 2, stimulation to the subject 2 can be reduced. The sidewall 246 may have a structure that is connectable to the support member 220.

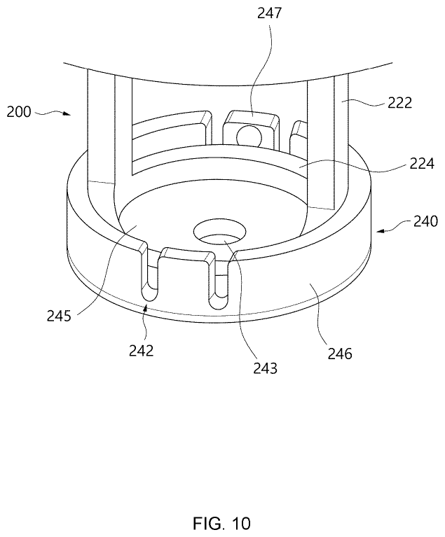

Specifically, the sidewall 246 may include a second connection portion 247, and the second connection portion 247 may be coupled to the first connection portion 224 (see FIG. 10). Specifically, the second connection portion 247 may include a protrusion 248, and the protrusion 248 may be coupled to at least a portion of the support member 220. One or more grooves may be formed in the sidewall 246. Preferably, two grooves may be formed in the sidewall 246. The two grooves may be formed in both sides of the second connection portion 247. Since the grooves are formed in both sides of the second connection portion 247, the second connection portion 247 may have a structural elastic force.

Referring to FIG. 8, the contact portion 242 may include a contact surface 244. The contact surface 244 may be formed at a lower side of the contact portion 242. The contact surface 244 may be formed at the lower side of the contact portion 242 to be in direct contact with the target 1 and/or the subject 2. In addition, the contact surface 244 may be formed to have a predetermined area or more. Since the contact surface 244 is formed to have the predetermined area or more, the contact surface 244 increases a contact area of the subject 2, thereby preventing the laser irradiation device 100 from being inclined when in contact with the subject 2. In addition, the contact surface 244 may be formed to have a predetermined angle with an axis of an irradiation direction of the pulsed beam. For example, the predetermined angle is preferably a right angle but is not limited thereto. Since the contact surface 244 is formed to have the predetermined angle with the axis of the irradiation direction of the pulsed beam, when the contact surface 244 is in contact with the target 1 and/or the subject 2 by a predetermined area or less, a direction of the pulsed beam irradiated onto the target 1 may be determined. In addition, a distance by which the pulsed beam is irradiated from a laser irradiation module to the target 1 may also be set to be constant.

Referring to FIG. 9, each portion of the contact member 240 may have a preset thickness.

Specifically, the contact portion 242 may have a first thickness T1. Here, the first thickness T1 may be determined in consideration of a position of the laser generation module 120. The first thickness T1 may be determined such that the contact surface 244 is positioned within a focal depth range.

In addition, a thickness from the contact surface 244 to an upper end of the contact member 240 may be set as a second thickness T2. The second thickness T2 may be determined such that, when the contact member 240 is mounted on the support member 220, the contact surface 244 is positioned at a focal length or within a focal depth range of the pulsed beam 5.

In addition, the through-hole 243 may have a predetermined width. As will be described below in detail, since the through-hole 243 has a width determined to be less than or equal to a predetermined length, even when the contact member 240 presses the target 1 or the subject 2, the target 1 may be positioned within the focal depth range of the pulsed beam.

FIG. 10 illustrates a coupling structure of the guide part of the laser irradiation device according to one embodiment.

Referring to FIG. 10, the guide part 200 according to one embodiment may be formed by coupling the support member 220 and the contact member 240.

The support member 220 may include a support structure 222 disposed at one side of the housing 101 and the first connection portion 224 formed by extending one end of the support structure 222. The support structure 222 may have a rod shape or a bar shape. In addition, the first connection portion 224 may have a rim shape. The first connection portion 224 may have a shape corresponding to the contact portion 242. The first connection portion 224 may have a shape corresponding to the sidewall 246. An outer diameter of the first connection portion 224 may be formed to have a size corresponding to an inner diameter of the sidewall 246. Since the outer diameter of the first connection portion 224 is formed to have the size corresponding to the inner diameter of the sidewall 246, the contact member 240 may be fixed to the support member 220.

Here, the support member 220 may have a predetermined strength so as to not be deformed even when a predetermined external force is applied thereto. When the laser irradiation device 100 is used, a user may press the laser irradiation device 100 against the target 1 or the subject 2 in order to fix an irradiation position of the pulsed beam. Even in this case, the support member 220 may have a strength greater than or equal to a predetermined strength such that an irradiation position or an irradiation distance of the pulsed beam is not changed.

The support member 220 and the contact member 240 may be coupled in a snap-fit form. When the first connection portion 224 is inserted, the second connection portion 247 having a structural elastic force due to the grooves at both sides thereof may be moved outward and then repositioned to fix the first connection portion 224. That is, the first connection portion 224 may be fixed between the protrusion 248 and the contact portion 242. In this case, a distance between an upper surface of the contact portion 242 and a lower portion of the protrusion 248 is designed to correspond to a thickness of the first connection portion 224, thereby preventing movement of the first connection portion 224.

The protrusion 248 may have a semispherical shape. Since the protrusion 248 has the semispherical shape, the first connection portion 224 may be smoothly inserted along a curved surface of the protrusion 248. Alternatively, one area of the protrusion 248 may be formed to have a curved surface, and the other area thereof may be formed to have a flat surface. An area of the protrusions 248 adjacent to the contact portion 242 may be formed as a flat surface, and an area thereof spaced apart from the contact portion 242 may be formed as a curved surface. Accordingly, the first connection portion 224 may be smoothly inserted along the curved surface of the protrusion 248 and then fixed by the flat surface thereof.

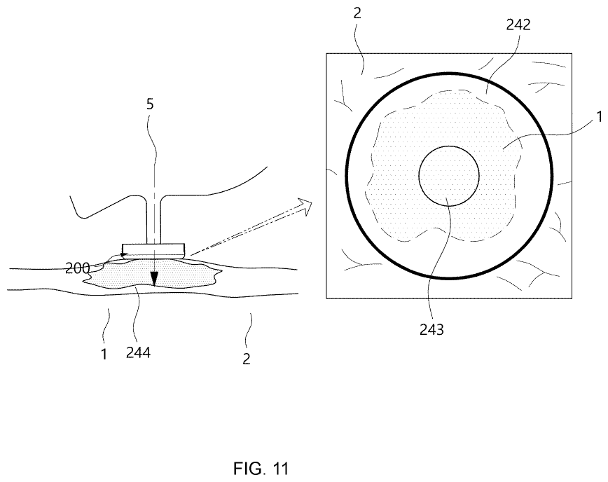

FIG. 11 illustrates the contact member in contact with a subject being observed in a direction in which a pulsed beam is irradiated according to one embodiment.

The contact member 240 according to one embodiment may be in contact with the subject 2. Specifically, the contact surface 244 of the contact portion 242 may be in contact with the subject 2 or the target 1 by a predetermined area or more. When the contact portion 242 is in contact with the subject 2 and/or the target 1, the laser irradiation device 100 may irradiate the pulsed beam 5 through the through-hole 243 formed in the contact portion 242 and may induce plasma ablation. Here, the pulsed beam 5 is preferably irradiated toward the central portion of the through-hole 243, but the present invention is not necessarily limited thereto as described above.

From the viewpoint of use of the laser irradiation device 100 according to the embodiment, it is important to accurately determine an irradiation point of the pulsed beam 5. The spot size of the pulsed beam 5 is in a range of 0.001 mm and 10 mm as described above. Therefore, since it is materially difficult for a user to visually identify the irradiation point of the pulsed beam 5 accurately, it is necessary to assist the user using the laser irradiation device 100 in identifying the irradiation point of the pulsed beam 5.

The contact member 240 according to the embodiment may be made of a transparent material. The contact member 240 may be made of a transmissive or semi-transmissive material to assist the user in identifying the irradiation point of the pulsed beam 5. That is, the user may visually observe the subject 2 through the contact member 240 to set the irradiation point of the pulsed beam 5.

In summary, the user may visually observe the subject 2 through the contact member 240 and may align the laser irradiation device 100 such that the target 1 is positioned in the through-hole 243. The user may arrange the laser irradiation device 100 such that a predetermined area or more of the contact surface 244 is in contact with at least a portion of the target 1 and/or the subject 2 formed around the target 1, thereby irradiating the pulsed beam 5. Accordingly, the user can accurately match the irradiation point of the pulsed beam 5 to the target 1.

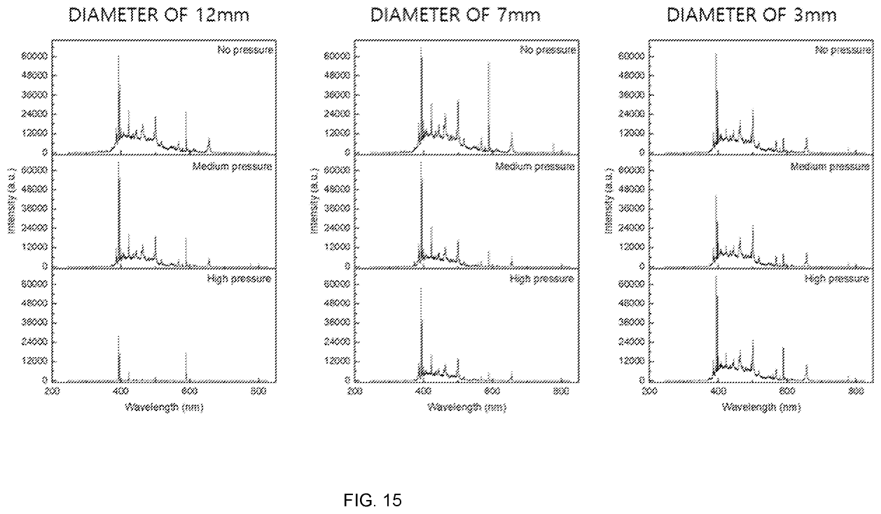

Here, predetermined pressure may be applied between the contact member 240 and the target 1 and/or the subject 2 in order for the predetermined area or more of the contact surface 244 to be in contact with the subject 2 formed around the target 1 or for other reasons. As described above, when the guide frame 3 (see FIG. 4) according to the related art presses the subject 2, the subject 2 is pushed up more than necessary, which may have a negative influence on plasma ablation being uniformly induced. However, in the laser irradiation device 100 according to one embodiment, as will be described below in detail with reference to FIG. 15, even when the user presses the contact member 240 such that the predetermined area or more of the contact surface 244 is in contact with the subject 2, the target 1 is positioned within the focal depth range of the pulsed beam 5, thereby inducing uniform plasma ablation to acquire uniform spectrum data.

In the above, the structural configuration of the laser irradiation device 100 according to one embodiment has been described with reference to the drawings.

Hereinafter, the optical structure and function of the laser irradiation device 100 will be described with reference to FIG. 12.

FIG. 12 illustrates a path of a pulsed beam output from the laser irradiation device according to one embodiment together with a configuration of the laser irradiation device.

The laser generation module 120 generates a pulsed beam having a preset focal length to output the generated pulsed beam through the opening 104. Here, as described above, a pulsed beam 5 may have a focal depth range in a preset range with respect to a focus thereof along an axis of an irradiation direction of a laser. In addition, the laser generation module 120 may include the optical member 121. For example, the optical member 121 may be provided as a collimating lens to output a received focused beam as a collimated beam. In addition, the optical member 121 may be provided as a focus lens for changing a focal length of a laser into a specific distance. That is, the focal length or focal depth range of the pulsed beam 5 may be changed by the optical member 121.

The contact member 240 may be disposed at a preset position. Specifically, the contact member 240 may be disposed within the focal depth range of the pulsed beam 5. More specifically, since the contact surface 244 is disposed within the focal depth range of the pulsed beam 5, when the contact surface 244 is in contact with the target 1 and/or the subject 2, the target 1 may be positioned within the focal depth range of the pulsed beam 5. Here, the contact surface 244 may also be positioned at the focal length of the pulsed beam 5. In addition, the contact surface 244 may be disposed to be positioned within a half range of the focal depth range with respect to the focus of the pulsed beam 5.

The support member 220 may have a preset length such that the contact member 240 is positioned within the focal depth range of the pulsed beam 5. Alternatively, the support member 220 may have a preset length such that a distance from the optical member 121 to at least a portion of the contact member 240 corresponds to the focal length of the pulsed beam 5.

For example, when the laser generation module 120 is mounted such that the opening 104 is positioned at the same position as a start point (for example, the optical member 121) from which the pulsed beam 5 is output, a distance from the opening 104 to the contact surface 244 of the contact member 240 may correspond to the focal length of the pulsed beam 5 or a distance to at least one point within the focal depth range. In addition, when the support member 220 is mounted at the same position as the opening 104, the sum of a length of the support member 220 and the first thickness T1 (see FIG. 10) of the contact member 240 may correspond to the focal length of the pulsed beam or the distance to at least one point within the focal depth range.

A specific example will be described with reference to the drawings.

A length L3 of the guide part 200 may be determined in consideration of the focal length of the pulsed beam 5. Specifically, the length L3 of the guide part 200 may be determined such that a distal end of the contact member 240 in an irradiation direction of a laser is positioned within the focal depth range of the pulsed beam 5. For example, the length L3 of the guide part 200 may be determined such that the contact surface 244 is disposed at a position corresponding to a focus f of the pulsed beam 5. In addition, according to a mounting position in an accommodation space 102 of the laser generation module 120, a distance from the optical member 121 to the opening 104 may be a first distance L2.

For example, when the first distance L2 is predetermined, the length L3 of the guide part 200 may be determined such that the distal end of the contact member 240 is positioned within the focal depth range of the pulsed beam 5. In addition, even when the length L3 of the guide part 200 is predetermined, the first distance L2 may be determined such that the distal end of the contact member 240 is positioned within the focal depth range of the pulsed beam 5.

Furthermore, here, the length L3 of the guide part 200 and/or the first distance L2 may be determined in consideration of a coupling structure between the support member 220 and the housing 101.

For example, according to various embodiments, the support member 220 and the housing 101 may be formed such that the length L3 of the guide part 200 and the first distance L2 overlap each other. Even in this case, the length L3 of the guide part 200 and/or the first distance L2 may be determined such that a position of at least a portion of the contact member 240 is positioned within the focal depth range of the pulsed beam 5.

This may be represented by Expression as follows.

L3+L2≥focus distance of pulsed beam Expression 1:

That is, in order for the contact surface 244 to be disposed at the focal length of the pulsed beam 5, the sum of the first distance L2 and the length L3 of the guide part 200 should be at least greater than or equal to the focal length of the pulsed beam.

However, the focal length of the pulsed beam in the above Expression is merely an example, and various distances at which the contact surface 244 can be positioned within the focal depth range may be applied to the right term of the above Expression. For example, a distance from the laser irradiation module (specifically, the optical member) to a boundary of the focal depth range of the pulsed beam in an irradiation direction of a laser may be applied to the right term of the above Expression.

In addition, in another embodiment, the support member 220 may be positioned near the opening 104 and may have a first length L1. Furthermore, the contact portion 242 may have a first thickness T1.

The first length L1, the first distance L2, and the first thickness T1 may be set such that the contact surface 244 is positioned within the focal length or the focal depth range of the pulsed beam 5.

For example, when the first length L1 and the first distance L2 are predetermined, the first thickness T1 may be determined such that, when the contact member 240 is mounted on the support member 220, the contact surface 244 is positioned within the focal length or the focal depth range of the pulsed beam 5 output from the laser irradiation device 100. In particular, when the contact member 240 according to one embodiment is provided in a detachable form, the first thickness T1 may be determined in consideration of the first length L1 and the first distance L2. Alternatively, when the first distance L2 or the first thickness T1 is predetermined, the first length L1 may be determined to correspond thereto. Alternatively, when the first length L1 and the first thickness T1 are predetermined, the first distance L2 may be determined to correspond thereto. In particular, when the guide part 200 is mounted on the housing 101 as a separate member, the first distance L2 may be set to correspond to a length of the guide part 200.

In addition, here, the first distance L2, the first thickness T1, and/or the first length L1 may be determined in consideration of the coupling structure between the support member 220 and the housing 101. For example, according to various embodiments, as shown in the drawings, the support member 220 and the housing 101 may be formed such that the first length L1 and the first distance L2 overlap each other. Even in this case, the first distance L2, the first thickness T1, and/or the first length L1 may be determined such that a position of at least a portion of the contact member 240 is positioned within the focal depth range of the pulsed beam 5. This may be represented by Expression as follows.

L1+L2+T1≥focus distance of pulsed beam

Setting the target 1 to be positioned within the focal depth range of the pulsed beam 5 in the laser irradiation device 100 according to the embodiment is an important factor in terms of whether plasma ablation is induced.

Table 2 is a table showing whether plasma ablation is induced according to an irradiation distance of a pulsed beam.

TABLE 2

| | Irradiation distance (mm) | | | | | |
|---|---|---|---|---|---|---|
| | 30.12 | 31.12 | 32.12 | 33.12 | 34.12 | 35.12 |
| Distance | 3% | 0 | 3% | 6% | 10% | 13% |
| deviation (%) | | | | | | |
| Generation | 50/50 | 50/50 | 50/50 | 17/50 | 6/50 | 0/50 |

Table 2 is a table in which the focal length of the pulsed beam 5 is set to 31.12 mm, and an irradiation distance, which is a distance between the subject 2 and a start point from which the pulsed beam 5 is output, is changed to examine whether plasma ablation is generated. In this case, the subject 2 is skin of a human.

Referring to Table 2, as an irradiation distance of a pulsed beam became similar to a focal length of the pulsed beam, plasma was well induced, but as the irradiation distance of the pulsed beam became farther away from the focal length of the pulsed beam, plasma ablation tended to not be induced in a target.

Specifically, when a deviation between the focal length and the irradiation distance of the pulsed beam 5 was 3% or less, plasma ablation was generated in all of 50 pulsed beam irradiations, and when the deviation between the focal length and irradiation distance of the pulsed beam 5 was 6% or more, plasma ablation was not well induced. That is, when the irradiation distances of the pulsed beam were 30.12 mm, 31.12 mm, and 32.12 mm, plasma ablation was induced, and when the irradiation distances were 33.12 mm, 34.12 mm, and 35.12 mm, a desired degree of plasma ablation was not induced.

As described above, in the laser irradiation device 100 according to the embodiments, when the contact member 240 is in contact with the target 1 or the subject 2, the target 1 is allowed to be positioned within the focal depth range (or the focal distance) of the pulsed beam 5, thereby inducing uniform plasma ablation in the target 1.

Hereinafter, even when pressure is applied to the laser irradiation device 100, the structure of the contact member for allowing the target 1 to be positioned within the focal depth range of the pulsed beam 5 will be described.

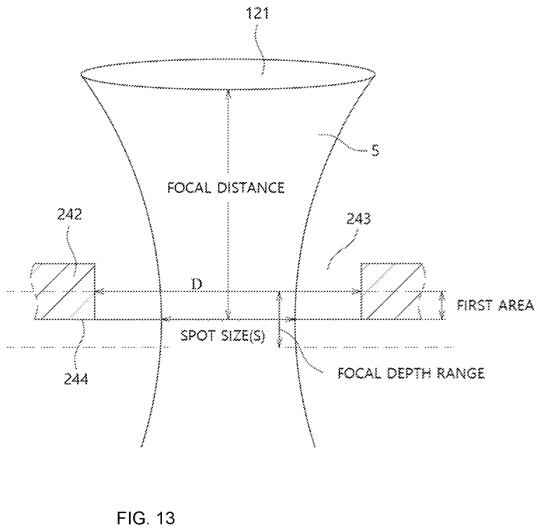

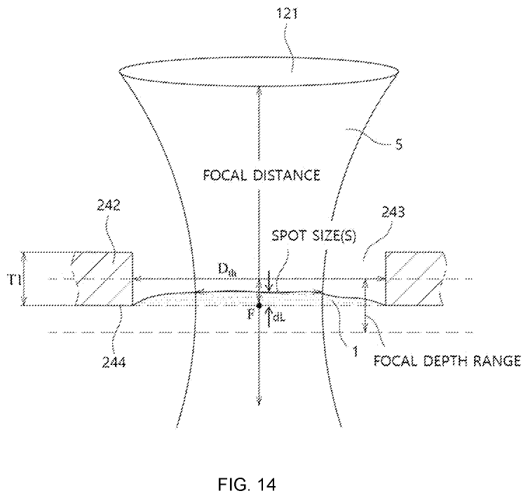

FIGS. 13 and 14 illustrate embodiments of the laser irradiation device 100 according to various use conditions.

Specifically, FIG. 13 illustrates an irradiation path of a pulsed beam when there is no target, and FIG. 14 illustrates an irradiation path of a pulsed beam when the contact member presses a target.

An irradiation path of a pulsed beam 5 will be described with reference to FIG. 13.

The pulsed beam 5 output from the optical member 121 may travel through the through-hole 243. Here, a diameter D of the through-hole 243 may be set in consideration of a spot size S at a focal length of the pulsed beam 5. The diameter D of the through-hole 243 may be set to be greater than the spot size S at the focal length of the pulsed beam 5. This is because, when the diameter D of the through-hole 243 is less than or equal to the spot size S at the focal length of the pulsed beam 5, due to a process error or an optical path alignment error, a portion of the pulsed beam 5 does not pass through the contact member 240, and thus energy of the pulsed beam 5 is not transmitted to a target 1, which may fail to induce plasma ablation. For example, the diameter D of the through-hole 243 may be set to be greater than twice the spot size S at the focal length of the pulsed beam 5.

In addition, the focal length of the pulsed beam 5 may correspond to a distance from the optical member 121 to the contact surface 244. That is, a focus of the pulsed beam 5 may be positioned in one area of the through-hole 243 corresponding to a position of the contact surface 244. A focal depth range of the pulsed beam 5 may be formed in a first range formed along an axis of an irradiation direction of the pulsed beam from the focus of the pulsed beam 5.

The contact member 240 may be disposed such that at least a partial area of the through-hole 243 is included in the focal depth range of the pulsed beam 5. Specifically, the contact member 240 may be disposed such that an area formed along an axis of an irradiation direction of a laser in the through-hole 243 is included within the focal depth range of the pulsed beam 5.

Here, the diameter D of the through-hole 243 may be set such that the target 1 is positioned within the focal depth range even when the target 1 and/or subject 2 are pressed by the contact member 240. As shown in FIG. 14, when the target 1 is pressed by the contact member 240, the target 1 may protrude by a protrusion distance dL in a direction opposite to a pressing direction. When the target 1 is pressed with strong pressure, the protrusion distance dL may be increased, and when the target 1 is pressed with weak pressure, the protrusion distance dL may be decreased. In addition, when the diameter D of the through-hole 243 is increased, the protrusion distance dL may be increased, and as the diameter D of the through-hole 243 is decreased, the protrusion distance dL may be decreased.

Since a use mode of the laser irradiation device is different according to users, the diameter D of the through-hole 243 should be set such that plasma ablation is induced even when the target 1 is pressed with any level of pressure through the laser irradiation device. That is, the diameter D of the through-hole 243 should be set such that a difference between a focal length and an irradiation distance is within 1 mm as shown in experimental results of Table 1 even when any level of pressure is applied to the target 1. That is, the diameter D of the through-hole 243 should be set such that the protrusion distance dL is 1 mm or less even when any level of pressure is applied. In other words, the diameter D of the through-hole 243 should be set so that a deviation between the focal length and the irradiation distance is 3% or less.

Table 3 is a table in which a protrusion distance of a target according to a diameter of a through-hole and pressure applied to the target is tested.

TABLE 3

| Diameter of | Protrusion distance (mm) | | |
|---|---|---|---|
| through-hole | Contact | Medium pressure | High pressure |
| 12 mm | 0 | 1.02 | 1.54 |
| 7 m | 0 | 0.47 | 0.96 |
| 3 mm | 0 | 0.16 | 0.24 |

Referring to Table 3, each of the contact members 240 in which through-holes have diameters of 3 mm, 7 mm, and 12 mm was provided and mounted, and in a case in which contact was made without pressure, in a case in which medium pressure is applied, and in a case in which high pressure is applied, a protrusion distance for each case was measured.

Here, high pressure refers to a level of pressure at which a human feels severe pain, contact refers to contact without pressure, and medium pressure refers a level of medium pressure when contact is made with high pressure.

When the diameter D of the through-hole 243 is 12 mm, a portion of the target 1 protrudes by 1.02 mm in a case in which medium pressure is applied, and a portion of the target 1 protrudes by 1.54 mm in a case in which high pressure is applied.

When the diameter D of the through-hole 243 is 7 mm, a portion of the target 1 protrudes by 0.47 mm in a case in which medium pressure is applied, and a portion of the target 1 protrudes by 0.96 mm in a case in which high pressure is applied.

When the diameter D of the through-hole 243 is 3 mm, a portion of the target 1 protrudes by 0.16 mm in a case in which medium pressure is applied, and a portion of the target 1 protrudes by 0.24 mm in a case in which high pressure is applied.

The diameter D of the through-hole 243 may be set to 7 mm or less. When the diameter D of the through-hole is set to 12 mm, even in a case in which medium pressure is applied, there may be a case in which the protrusion distance dL exceeds 1 mm, and plasma ablation is not induced according to Table 1.

Preferably, the diameter D of the through-hole 243 may be set to 3 mm or less. When the diameter D of the through-hole is 7 mm and high pressure is applied, the protrusion distance dL is 0.96 mm in a case in which high pressure is applied. Therefore, the protrusion distance dL may exceed 1 mm according to a use mode of a user and the elasticity of the subject 2 which is skin, and thus, when the diameter D of the through-hole 243 is set to 3 mm or less, stable plasma ablation can be induced.

A relationship between the diameter of the through-hole and the induction of plasma ablation can be further confirmed through FIG. 15 and Table 4.

FIG. 15 shows spectrum data according to a diameter of a through-hole and pressure, and Table 4 shows the sum of areas of spectrum areas according to a diameter of a through-hole and pressure.

TABLE 4

| | Sum of spectrum areas | | |
|---|---|---|---|
| Diameter | Contact | Medium pressure | High pressure |
| 3 mm | 1345790 | 1205930 | 1362190 |
| 7 m | 1902570 | 849927 | 623706 |
| 12 mm | 1717990 | 956843 | 55778 |

FIG. 15 and Table 4 show data measured by irradiating a laser onto the same target through a spectrum analysis system 1000 to induce plasma ablation, replacing and mounting contact members having through-holes with different diameters, and changing pressure.

Referring to FIG. 15 and Table 4, when the diameter of the through-hole 243 of the contact member 240 is 3 mm, in a case in which contact is made, in a case in which medium pressure is applied, and in a case in which high pressure is applied, all the cases have almost similar spectrum data. In addition, the sums of the areas of the spectrum areas also do not have a large deviation.

When the diameter of the through-hole 243 of the contact member 240 is 7 mm, in both cases in which medium pressure is applied and a case in which high pressure is applied, intensity for each wavelength is decreased as compared with a case in which contact is made. As a result, the sum of the areas of the spectrum areas is also decreased. However, since shapes of graphs of peak wavelength bands and relative peak sizes are similar, it is possible to determine whether a target is abnormal through data normalization.

When the diameter of the through-hole 243 of the contact member 240 is 12 mm, in both cases in which medium pressure is applied and a case in which high pressure is applied, intensity for each wavelength is decreased as compared with a case in which contact is made. In particular, in a case in which high pressure is applied, since a shape of a graph cannot be identified, it is impossible to determine whether the target is abnormal.

Accordingly, when the diameter D of the through-hole 243 is 7 mm or less, the spectrum analysis system 1000 may determine whether the target is abnormal even when high pressure is applied. In addition, when the diameter D of the through-hole 243 is 3 mm or less, data distortion is small even when any level of pressure is applied, and thus, the determination accuracy of the spectrum analysis system 1000 can be improved.

Hereinafter, a method of inducing induced light by irradiating a pulsed beam 5 onto a target 1 using a laser irradiation device 100 according to one embodiment will be described.

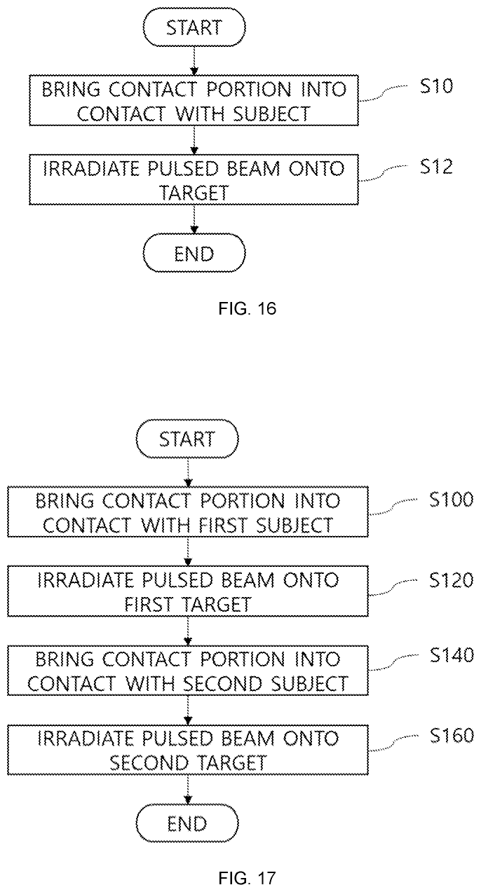

FIG. 16 illustrates a method of generating induced light in a single target using a laser irradiation device according to one embodiment.

Referring to FIG. 16, the method of irradiating a pulsed beam onto a single target may include bringing a contact portion into contact with a subject (S10) and irradiating a pulsed beam 5 onto a target 1 (S12).

Methods of irradiating a pulsed beam according to various embodiments disclosed in the present application may be performed by a medical robot or may be performed by a user using a laser irradiation device 100 of the present application. However, in the following description of the present application, it will be mainly described that various methods of irradiating a pulsed beam disclosed in the present application are performed by the user, but the present invention is not limited thereto. In addition, a method of irradiating a pulsed beam according to one embodiment of the present application performed by a medical robot is provided in the form of a program for controlling a medical robot to drive the method and a computer-readable electronic recording medium storing the program.

First, a user may bring at least a portion of a contact member 240 into contact with a subject 2 (S10). Specifically, the user may align a laser irradiation device 100 on the subject 2 such that a predetermined area or more of a contact surface 244 is in contact with the subject 2. Here, the user may align the laser irradiation device 100 on the subject 2 such that the target 1 corresponds to a through-hole 243. In this case, the user may press the subject 2 with predetermined pressure through the contact member 240. Specifically, the user may press the subject 2 through the contact member 240 with a force less than or equal to first pressure such that the predetermined area or more of the contact surface 244 is in contact with an area around the target 1. However, this is not necessary, and when it is determined that the predetermined area or more of the contact surface 244 is in contact with the area around the target 1 without additional pressure, such as when the target 1 is the same as a portion of the flat subject 2, such a process may be omitted.

When the predetermined area or more of the contact surface 244 is in contact with the subject around the target 1, an irradiation point of a pulsed beam 5 may be specified as the target 1, and an irradiation distance of the pulsed beam 5 may be determined from a laser generation module 120 within a focal depth range. Accordingly, it may be regarded that the preparation for inducing plasma ablation in the target 1 is completed.

Thereafter, the user may induce plasma ablation by operating the laser irradiation device 100 to irradiate the pulsed beam 5 onto the target 1 (S12). When the pulsed beam 5 is irradiated onto the target 1, plasma ablation may be induced in at least a portion of the target 1, and induced light may be generated from the plasma ablation. A data analysis device 1001 may analyze spectrum data about the induced light to acquire medical information about the subject 2 and/or the target 1.

FIG. 17 illustrates a method of generating induced light in a plurality of targets using a laser irradiation device according to one embodiment.

When a data analysis device 1001 according to one embodiment determines medical information about a target 1, spectrum data in various formats may be required according to a method of training a machine learning model.

For example, when the machine learning model determines medical information about a first object to be analyzed, the machine learning model requires not only spectrum data about the first object but also spectrum data about a second object different from the first object. For example, the first object and the second object may be distinguished according to shapes of tissues. Specifically, the first object may be skin tissue suspected of having skin cancer, and the second object may be skin tissue determined to be normal. In addition, the first object and the second object may be distinguished according to regions in which tissues are present. Specifically, the first object may be tissue present in a hand, and the second object may be tissue present in a foot. In addition, the first object and the second object may be distinguished in various manners according to a method of training the machine learning model.

More specifically, when the machine learning model is trained with combined spectrum data in which the spectrum data about the first object and the spectrum data about the second object are combined, in order to acquire data input to the machine learning model, both of the spectrum data about the first object and the spectrum data about the second object should be acquired. In addition, when a variety of spectrum data is used, it will be essential that plasma ablation should be uniformly induced.

In this case, the user needs to generate induced light for each object by irradiating the pulsed beam 5 not only onto the first object but also onto the second object.

Referring to FIG. 17, the method of generating induced light in a plurality of targets using a laser irradiation device according to one embodiment may include bringing a contact portion into contact with one area of a first subject (S100), irradiating a pulsed beam onto a first target (S120), brining the contact portion into contact with one area of a second subject (S140), and irradiating a pulsed beam onto a second target (S160).

Operation S120 of irradiating the pulsed beam onto the first target to generate induced light may be substantially similar to that of the method of irradiating a pulsed beam to a single target of FIG. 16. However, here, the first subject may be a subject related to the first target. For example, the first subject may refer to a subject having the same physical properties as the first target. As another example, the first subject may be present in the same body part as the first target. As a specific example, when the first target is tissue suspected of being skin cancer, the first subject may be skin tissue formed around the first target.

In addition, the first target and the second target may be included in the same subject or may be included in different subjects.

When induced light is generated for the first target, a user may irradiate a pulsed beam to the second target to generate induced light in the second target. An operation of irradiating a pulsed beam onto the second target to generate induced light is similar to a process of irradiating a pulsed beam onto the first target to generate induced light, but since there are some differences, the differences will be mainly described.

When the user brings the contact member 240 into contact with the first subject 2, first pressure may be applied to the first subject 2. However, when the user brings the contact member 240 into contact with the second subject 2, second pressure different from the first pressure may be applied to the second target.

Here, even in a case in which the first pressure and the second pressure are respectively applied to the first subject and the second subject through a contact member 240, the properties of plasma ablation induced in the first target and the second target may be uniform.

Specifically, a through-hole 243 according to one embodiment may have a width formed to be less than or equal to a preset length. Accordingly, in both of a case in which the first pressure is applied to the first subject and a case in which the second pressure is applied to the second subject, a height by which the first target and the second target are pushed up is maintained within the above-described first area (that is, within a focal depth range of a pulsed beam).

That is, when a pulsed beam 5 output from a laser irradiation device 100 according to one embodiment is applied to the first target and the second target, since a difference in irradiation path of the pulsed beam 5 is substantially insignificant, plasma ablation induced in the first target and the second target may also be substantially uniform. In addition, a data analysis device 1001 may acquire spectrum data about induced light generated in each of the plasma ablations uniformly induced in the first target and the second target and may combine and analyze pieces of spectrum data to acquire medical information about the first target and/or the second target.

As described above, while the embodiments have been described with reference to specific embodiments and drawings, various modifications and alterations may be made by those skilled in the art from the above description. For example, desired results may be achieved although the embodiments of the present invention are performed in other sequences different from the descriptions, and/or the elements, such as a system, a structure, a device, a circuit, and so on, are combined or assembled in other ways different from the descriptions, or replaced or substituted with other elements or their equivalents.

Therefore, other implementations, other embodiments, and equivalents of the appended claims may be included in the scope of the appended claims.

What is claimed is:

1. A laser irradiation device for inducing plasma ablation in skin, the laser irradiation device comprising:
a housing which has an accommodation space formed therein and an opening formed in one side thereof;
a laser module which is positioned in the accommodation space and outputs a pulsed beam having a predetermined focal length and a predetermined focal depth range through the opening;
a guide member positioned at one side of the housing; and
a contact member which is mounted on the guide member and includes a contact portion having a through-hole, through which the pulsed beam passes, formed therein,
wherein:
the contact portion is in contact with a subject to assist in fixing an irradiation point of the pulsed beam;
one end of the contact portion is positioned within the focal depth range such that plasma ablation is induced in at least a portion of the subject; and
a diameter of the through-hole having a circular shape is formed to be less than or equal to a preset length such that the at least a portion of the subject is maintained within the focal depth range even when the contact member presses the subject.

2. The laser irradiation device of claim 1, wherein the diameter of the through-hole is set such that the subject is maintained to be 3% or less of the focal length even when the contact member presses the subject.

3. The laser irradiation device of claim 1, wherein the diameter of the through-hole is set such that the subject protrudes by 1 mm or less when the contact member presses the subject.

4. The laser irradiation device of claim 1, wherein the diameter of the through-hole is 7 mm or less.

5. The laser irradiation device of claim 1, wherein the diameter of the through-hole is 3 mm or less.

6. The laser irradiation device of claim 1, wherein the diameter of the through-hole is greater than a spot size of the pulsed beam.

7. The laser irradiation device of claim 1, wherein:
the focal depth range is defined as a preset range formed along an axis of an irradiation direction of the pulsed beam from a focus of the pulsed beam; and
the one end of the contact portion is positioned within a half range of the focal depth range based on the focus.

8. The laser irradiation device of claim 7, wherein the one end of the contact portion is positioned at the focus.

9. The laser irradiation device of claim 1, wherein the contact member has a thickness of 0.5 mm to 1.5 mm.

10. The laser irradiation device of claim 9, wherein:
a distance from one end of the guide part to a distal end of the contact member is defined as a first length;
a distance between the opening and a start point from which the pulsed beam is output is defined as a second length; and
a sum of the first length and the second length is set to be greater than or equal to the focal length of the pulsed beam so that the contact portion is maintained within the focal depth range even when the guide part and the housing are coupled to overlap each other.

11. The laser irradiation device of claim 1, wherein the contact member is made of a light-transmitting material.

12. The laser irradiation device of claim 1, wherein the contact member is formed to be detachable from and attachable to the guide member.

13. The laser irradiation device of claim 1, wherein:
the contact member includes a sidewall on which a connection portion is formed; and
the connection portion is coupled to the guide member in a snap-fit form.

14. A contact member which is used in a laser irradiation device for inducing plasma ablation in skin, the contact member comprising:
a contact portion which has an opening having a circular shape, through which a pulsed beam, which is output from the laser irradiation device and has a preset focal length and a preset focal depth range, passes; and
a connection portion connected to the contact portion and connected to one end of the laser irradiation device,
wherein:
the contact portion is mounted at the one end of the laser irradiation device and is in contact with a subject to assist in fixing an irradiation point of the pulsed beam;
one end of the contact portion is positioned within the focal depth range such that plasma ablation is induced in at least a portion of the subject when the contact portion is mounted on the laser irradiation device; and
a diameter of the opening is formed to be less than or equal to a preset length such that the at least a portion of the subject is maintained within the focal depth range even when the contact member presses the subject.

15. A method of inducing plasma ablation using a laser irradiation device including a laser module configured to output a pulsed laser having a preset focal length and a preset focal depth range through an opening formed in a housing, and a contact member including a contact portion which has a through-hole having a circular shape, through which the pulsed laser passes, formed therein, the contact portion being disposed within the focal depth range, the method comprising:
bringing the contact portion into contact with a first subject;
irradiating a pulsed beam onto the first target;
receiving light induced in the first target;
bringing the contact portion into contact with a second subject;
irradiating the pulsed beam onto a second target; and
receiving light induced in the second target,
wherein, in order for a position of the first target lifted by pressure when the contact portion is in contact with the first subject and a position of the second target lifted by pressure when the contact portion is in contact with the second subject to be maintained within the focal depth range, a diameter of the through-hole is formed to be less than or equal to a preset length.
